(19) United States
(12) Patent Application Publication
(10) Pub. No.: US 2026/0283514 A1
(43) Pub. Date: Sep. 24, 2026

(54) ASPIRATION SYSTEM FOR ASPIRATING A BIOLOGICAL SAMPLE

(71) Applicant: Beckman Coulter, Inc., Brea, CA (US)

(72) Inventors: Bart J. Wanders, Brea, CA (US); Marco Zuleta, Brea, CA (US)

(21) Appl. No.: 19/572,113

(22) Filed: Mar. 19, 2026

Related U.S. Application Data
(60) Provisional application No. 63/775,696, filed on Mar. 21, 2025.

(51) Int. Cl. A61B 5/15

(52) U.S. Cl. CPC A61B 5/150099

(57) ABSTRACT

An aspiration system for a biological analyzer includes: an aspiration needle configured to aspirate a blood sample from a specimen vial, wherein the aspiration needle includes a distal aperture; a motor operatively connected to the aspiration needle, the motor configured to control an elevation of the aspiration needle; an air pump configured to expel air from a distal region of the aspiration needle; and a controller configured to detect a change in pressure associated with the distal aperture of the aspiration needle.

### CROSS REFERENCE TO RELATED APPLICATIONS

This application claims the benefit of U.S. Provisional Application No. 63/775,696, filed on Mar. 21, 2025, the entirety of which is incorporated by reference herein.

### BACKGROUND

Generally, this application relates to aspiration systems that operate with (or in) instruments/analyzers used for analysis of a biological sample, such as human blood. One such aspiration system includes an aspiration needle. An aspiration system can aspirate the biological sample to form aliquots that can be analyzed by one or more processes in the instrument/analyzer. Such processes include imaging of particles in the sample, measuring the impedance of the biological sample, fluorescence, molecular testing, immunoassays, DNA sequencing, or the like.

An aspiration system may have a probe that is inserted into a vial where the biological sample is retained. The vial can have a stopper that is made from, for example, rubber. The probe includes a lumen and a distal aperture through which fluids in the vial are drawn. To position the distal aperture of the probe into the biological sample, the tip of the probe may be pushed through the stopper. Certain known probes have grooves on the exterior surface of the probe. Such grooves extend along the vertical length of the probe. The grooves assist in aspirating by creating open regions through the stopper. The open regions allow the passage of air between the vial and the external environment, such that pressure in the vial can be equilibrated to the pressure in the external environment (e.g., atmospheric pressure).

However, the grooves can deteriorate the stopper, especially after multiple aspirations from the same vial. Such deterioration can result in particles breaking off from the stopper (e.g., “coring”). These particles can be aspirated with the biological sample and may cause negative effects in the analyzer. For example, the particles can cause blockages or impact the accuracy of the analysis processes.

Additionally, aspirators are moved to a predetermined elevation in the vial, such as an elevation where the distal aperture of the probe is close to the bottom of the vial. With certain known aspiration systems, there is no way to know the exact elevation of the probe tip. To make sure that the biological sample is aspirated through the distal aperture of the probe, the elevation of the probe is lowered such that its estimated elevation results in the distal aperture being relatively deep in the biological sample. By doing this, portions of the biological sample can accumulate on the exterior of the probe in the grooves. This accumulation can lead to waste of the biological sample. It can also result in carry-over of the biological sample to subsequent vials.

Further, if the tip of the probe is lowered too far, it can puncture the bottom of the vial. Therefore, the lowest elevation of the distal aperture may be conservatively set to avoid this problem. Yet, this can lead to a substantial height of the sample not being available for aspiration, thereby leading to more waste of the biological sample.

Moreover, sometimes the biological sample in the vial may be too low (a “short sample”). If the volume of the biological sample aspirated is less than the required volume for a particular analysis process, this can could throw off calculations by the analysis process, thereby resulting in erroneous analysis.

There is therefore a need for aspiration systems that improve performance with respect to one or more of the aforementioned issues.

### SUMMARY

According to an embodiment, an aspiration system includes: an aspiration needle configured to aspirate a blood sample from a specimen vial including a gas having a pressure different from atmospheric pressure; a motor operatively connected to the aspiration needle, the motor configured to control an elevation of the aspiration needle; an air pump configured to expel air from a distal region of the aspiration needle; and a controller configured to detect a change in pressure associated with the distal aperture of the aspiration needle. The specimen vial may further include a gas that has a pressure greater or less than the environmental pressure. The aspiration system may equilibrate the pressure of the gas to be the environmental pressure. The air pump may be configured to control a volume of air pulled through the aspiration needle. The air pump may be a piezoelectric air pump. The controller may be configured to: determine that the distal aperture of the aspiration needle has reached the blood sample by detecting an increase in pressure; and allow aspiration to occur when the distal aperture of the aspiration needle is below a surface of the blood sample. The aspiration needle may include a chamber in fluid communication with the air pump. The controller may include a pressure transducer configured to measure a pressure within the chamber. The aspiration needle may include a probe. The controller may be configured to calculate a height of the blood sample based on a number of motor steps used to lower the probe to the blood sample. The controller may be configured to detect a short-sample condition when an elevation of the probe exceeds a predetermined elevation based on a predetermined volume of the blood sample. The aspiration system may further include a vent configured to allow pressure equilibration between a pressure within the aspiration needle and atmospheric pressure. The vent may be integrated with the air pump. The controller may be configured to calculate a required vacuum in the aspiration needle to aspirate or dispense a specific volume of the blood sample when the pressure is equilibrated. The aspiration system may further include a membrane between the chamber and the distal aperture of the aspiration needle, wherein the membrane is gas-permeable. The membrane may not be substantially permeable by the blood sample or a cleaning fluid. The probe may have an exterior surface without grooves. The controller may be configured to detect when the distal aperture of the aspiration needle is in a stopper on the specimen vial according to a detected increase in pressure. The controller may be configured to detect when the distal aperture of the aspiration needle has passed through the stopper and into a region of air within the specimen vial above the blood sample. The controller may be configured to operate the air pump to bring the pressure in the region of air in the vial to atmospheric pressure. The controller may be configured to cause the air pump to generate puffs of air in order to detect pressure.

According to an embodiment, a method for delivering samples includes: providing an aspiration needle for aspirating a blood sample from a specimen vial; controlling, with a controller, an elevation of the aspiration needle; expelling, with an air pump, air from a distal aperture of the aspiration needle; and detecting a change in pressure associated with the aspiration needle contacting the sample. The specimen vial may further include a gas that has a pressure greater or less than the environmental pressure. The method may further include equilibrating the pressure of the gas to be the environmental pressure. The method may further include controlling, with the controller, a volume of air pulled pushed by the air pump through the aspiration needle. The air pump may be a piezoelectric air pump. The method may further include: determining that the distal aperture of the aspiration needle has reached the blood sample by detecting an increase in pressure; and allowing aspiration to occur when the distal aperture of the aspiration needle is below a surface of the blood sample. The aspiration needle may include a chamber in fluid communication with the air pump. The method may further include measuring, with a pressure transducer included in the controller, a pressure within the chamber. The aspiration needle may include a probe. The method may further include: adjusting, with a motor, the elevation of the probe; and calculating a height of the blood sample based on a number of motor steps used to adjust the elevation of the probe. The method may further include detecting a short-sample condition when an elevation of the probe exceeds a predetermined elevation based on a predetermined height of a volume of the blood sample. The method may further include allowing pressure equilibration, with a vent, between a pressure within the aspiration needle and atmospheric pressure. The vent may be integrated with the air pump. The method may further include calculating, with the controller, a required vacuum in the aspiration needle to aspirate or dispense a specific volume of the blood sample when the pressure is equilibrated. A membrane may be provided to be located between the chamber and the distal aperture of the aspiration needle, wherein the membrane is gas-permeable. The membrane may not be substantially permeable by the blood sample. The probe may have an exterior surface without grooves. The method may further include detecting, with the controller, when the distal aperture of the aspiration needle is in a stopper of the specimen vial according to a detected increase in pressure. The method may further include detecting, with the controller, when the distal aperture of the aspiration needle has passed through the stopper and into a region of air within the specimen vial above the blood sample. The method may further include operating, with the controller, the air pump to bring the pressure in the region of air to atmospheric pressure. The method may further include causing, by the controller, the air pump to generate puffs of air in order to detect pressure.

### BRIEF DESCRIPTION OF SEVERAL VIEWS OF THE DRAWINGS

FIG. 1 is a schematic illustration, partly in section and not to scale, showing operational aspects of an exemplary aspiration system and vial/stopper containing a sample.

FIGS. 2A-2F are schematic illustrations, partly in section and not to scale, showing a sequence for aspirating a sample in a vial with the aspiration system, according to embodiments.

FIG. 2G is a schematic illustration, partly in section and not to scale, showing dispensation of an aspirated sample into a container, according to embodiments.

FIG. 3 is a flowchart for a method for aspirating and dispensing a sample using an aspiration system, according to embodiments.

FIG. 4 is a flowchart for a method of aspirating a sample using an aspiration system, according to embodiments.

The foregoing summary, as well as the following detailed description of certain techniques of the present application, will be better understood when read in conjunction with the appended drawings. For the purposes of illustration, certain techniques are shown in the drawings. It should be understood, however, that the claims are not limited to the arrangements and instrumentality shown in the attached drawings.

### DETAILED DESCRIPTION

Embodiments disclosed herein may enable an aspiration system to determine the pressure at the distal aperture of the probe at virtually any elevation above or within the stopper, vial, and/or biological sample contained within the vial. Furthermore, embodiments may enable an aspiration system to control pressure of air within the vial. Such control can allow more accurate aspiration of a desired volume of the biological sample, according to the principle of P₁V₁=P₂V₂. A pump is employed to control how much air is pulled from a chamber within the aspiration system. By creating a particular vacuum using the air pump, the desired volume of sample is aspirated, thereby equilibrating the pressure in the chamber to its original pressure. The pump also may include a vent to facilitate equilibration. Pressure in the chamber is measured by a transducer.

Embodiments herein describe a membrane that is gas-permeable, but substantially impermeable to liquid. The membrane can serve to mitigate deleterious effects of liquid entering into regions where there are electronic or sensitive mechanical components.

Further, a port is provided to wash the interior of the probe between sample. This can be advantageous because the aspiration system (all or part) may not be disposable.

Using similar principles and features of an aspiration system, fluid (e.g., air, biological sample, and/or cleaning liquid) can be dispensed into the vial.

To address the problem of coring, the probe on the aspiration system may have no grooves. This may substantially reduce friction between the probe and the stopper when the probe is inserted into the stopper. Further, without grooves, accumulation of the biological sample on the exterior surface of the probe can be reduced. Additionally, without grooves, there may be fewer gaps (or no gaps) between the stopper and the probe when the probe is inserted. When the probe is withdrawn from the stopper, a substantial amount of any accumulated biological sample on the exterior surface of the probe may be wiped and may remain in the vial.

For pressure equalization between the vial and exterior pressure (e.g., atmospheric pressure), the aspiration system can have a vent to let air in/out of the vial (e.g., through the air pump).

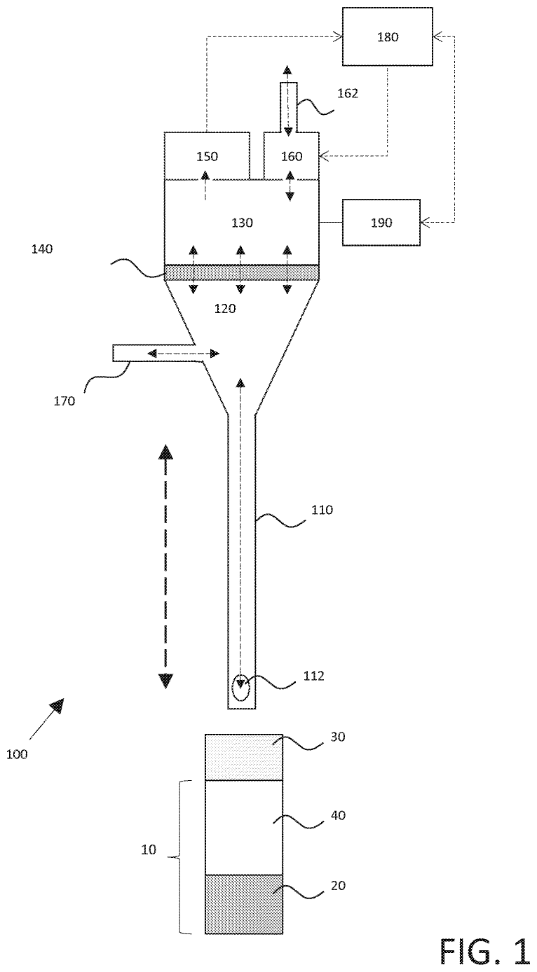

FIG. 1 illustrates an aspiration system 100, according to embodiments. The aspiration system 100 may not be disposable and may be used to aspirate or dispense fluid multiple times from a given vial 10, from multiple vials 10, or multiple times from each of a multiple number of vials 10. The aspiration system 100 may both aspirate and dispense fluid (e.g., gas and/or liquid, such as the biological sample 20 or cleaning fluid). When the aspiration system 100 aspirates a portion of the biological sample 20 in the vial 10, the aspirated portion of the biological sample 20 may form an aliquot that is delivered to an analysis process where the aliquot is analyzed. Exemplary analysis processes include hematology flow imaging, impedance measurement, fluorescence, molecular testing, immunoassays, DNA sequencing, or the like.

A vial 10 holds a sample 20 (e.g., biological sample, such as human blood) and has an aperture on the top of the vial 10. A stopper 30 (e.g., rubber stopper) engages with the vial 10 to seal the vial 10 and the sample 20 retained therein. The vial 10 has known dimensions, and accordingly, a given volume of the sample 20 may have a particular, known height within the vial 10. Therefore, it may be able to infer the volume of the sample 20 by measuring the height of the surface of the sample 20.

The aspiration system 100 may include a probe 110, a reservoir 120, a chamber 130, a membrane 140, a pressure transducer 150, a pump 160, a port 170, a controller 180, and/or a motor 190. In FIG. 1, the broken lines with arrowheads within the aspiration system 100 indicate directions of the flow of fluid. The dotted lines with arrowheads indicate electrical signals between the controller 180 and several other components (the pressure transducer 150, the pump 160, and the motor 190). The broken line with arrowheads to the left of the probe 110 indicates that the elevation of the probe 110 (or at least the distal tip of the probe) can be adjusted.

Components of the aspiration system 100 may be rigidly coupled with each other and/or integrated together, such that this collection of components will move together (e.g., move together up or down along the vertical dimension). Other components may (or may not) be connected in a non-integrated or a non-rigid manner. For example, the pump 160 and/or the transducer 150 may be connected to the chamber 130 via flexible tubing. The pump 160 and/or the transducer 150 may be maintained in particular location(s). The motor 190 may be connected to moving component(s) via movable linkage, but the motor 190 itself may be maintained in a particular location. The controller 180 may be electrically connected (either directly or indirectly) to moving components via flexible wires or cables, but the controller 180 itself may be maintained in a particular location.

The probe 110 is or is part of an aspiration needle. The probe 110 may include a material that is non-reactive to the biological sample 20 or other fluids presented during the aspiration/dispensation processes. Such a material can include stainless steel. The probe 110 may be a single integrated piece or a number of pieces coupled together. The probe 110 can have a pointed lower (or distal) region (not shown) that facilitates the probe 110 being pushed through the stopper 30. The probe 110 includes a lumen with a hollow interior region through which fluid flows. The probe 110 includes a distal aperture 112 that is proximate or in the lower region of the probe 110. The distal aperture 112 may be positioned on a lateral surface of the probe. Fluid can flow into or out of the lumen of the probe 110 through the distal aperture 112.

The reservoir 120 is in fluid communication with (e.g., rigidly coupled to) the proximal end of the probe 110. The reservoir 120 may be part of an aspiration needle. The reservoir 120 may contain a fluid that flows into or out of the probe 110. The reservoir 120 may narrow in width (e.g., taper) from a proximal end to a distal end of the reservoir 120. This arrangement may allow dispensation or aspiration of a wider range of liquid volumes. A port 170 can be in fluid communication with (e.g., coupled to) the reservoir 120. Fluid through the port 170 can be delivered to and/or drawn from the reservoir 120. One such fluid is cleaning liquid, which can be used to clean the reservoir 120 and probe 110 after an amount of usage. The port 170 may include flexible tubing. The port 170 may include a valve (not shown) to selectively provide the fluid to the reservoir 120. The valve may be controlled by the controller 180. The valve of the port 170 may be selectively closed to isolate the aspiration system 100 from external pressure via the port 170. The valve of the port 170 may be selectively controlled (e.g., by the controller 180) to deliver and/or draw fluid to/from the reservoir 120. The valve of the port 170 may be selectively closed (e.g., by the controller 180) to prevent fluid from flowing into or out of the reservoir 120.

The chamber 130 can be separated from the reservoir 120 by a membrane 140. The chamber 130 may be part of an aspiration needle. The chamber 130 may contain only gas during operation of the aspiration system 100. The membrane 140 may be part of an aspiration needle. The membrane 140 can be gas-permeable but not substantially liquid-permeable. The membrane 140 can discourage or prevent liquid in the reservoir 120 from passing into the chamber 130. In such a way, the transducer 150 and/or the pump 160 can remain substantially dry during operation of the aspiration system 100. The chamber 130 may be one large volume, or may be separated into chambers (that may be in fluid communication with each other).

The transducer 150 includes a pressure transducer that is in fluid communication with the interior of the chamber 130. The transducer 150 may be rigidly coupled to the chamber 130 or may be connected, for example, via flexible tubing. The transducer 150 generates an electrical signal having a level that corresponds to a sensed pressure (i.e., the pressure of gas in the chamber 130). The electrical signal is communicated to the controller 180, either directly or through intermediate componentry, such as an analog-to-digital converter (which may also be incorporated in a chip with the controller 180).

The pump 160 is an air pump and is in fluid communication with the interior of the chamber 130. The pump 160 may be rigidly coupled to the chamber 130, or the pump 160 may be coupled to the chamber via flexible tubing. The pump 160 is electrically connected to the controller 180, either directly or indirectly. The pump 160 receives signals from the controller 180 and either pumps air into or out of the chamber 130. This pumping causes fluid (gas and/or liquid) to be pushed into or pulled out of the probe 110. The volume of gas that the pump 160 pumps into or out of the chamber 130 causes a corresponding amount to be pushed into or pulled out of the probe 110. The pump 160 may be a piezoelectric pump. The pump 160 may further include a vent 162 that is in fluid communication with the external environment. The vent 162 may be selectively opened or closed via a valve controlled by the controller 180. The vent 162, when open, can allow the pressure in the chamber 130 to equilibrate with the pressure in the external environment (e.g., atmospheric pressure).

The motor 190 may include a rack and pinion system or a lead screw and nut system. The motor 190 may be a DC stepper motor, and may either directly or indirectly adjust the elevation of the probe 110. The motor 190 receives electrical signals from the controller 180, either directly or indirectly, to cause the motor 190 to move the probe 110 upwardly or downwardly. In the example of a DC stepper motor, the controller 180 can cause the motor 190 to increase or decrease a particular number of steps.

The motor 190 may provide feedback through electrical signals transmitted either directly or indirectly (e.g., via an analog-to-digital converter) to the controller 180. The feedback may indicate, for example, the resistance of the motor 190. The feedback is a sensed signal, and may be used by the controller 180 to determine a condition of the motor 190 (e.g., that the motor 190 is being blocked from increasing and/or decreasing steps.

The controller 180 may be a single controller (e.g., a processor, a microprocessor, a microcontroller or the like) or a collection of controllers operating together to perform specific operation(s). In the case of multiple controllers, they may be co-located (e.g., on a single chip) or distributed at different locations. The controller 180 executes a set of instructions stored on a non-transitory computer-readable medium to perform operations. The controller 180 can control components of the system (e.g., pump 160, vent 162, and/or motor 190) and receive sensed signals from components (e.g., transducer 150 and/or motor 190). The transducer 150 may be included in the controller 180.

The controller 180 may further control the positioning of the aspiration needle, such that it can aspirate samples from multiple vials, such as multiple vials in a carrier. One such carrier is a cassette-type carrier, and one such cassette-type carrier can be used to hold, for example, five to ten vials. Prior to aspiration, a cassette can be guided to a mixing station, where the samples in the vials get properly mixed. The aspiration needle or probe 110 can be mounted on an x-y gantry, which allows the probe 110 to be positioned along x-and/or y-axes. above a selected vial. After a sample is aspirated from a first vial, the controller 180 can move the aspiration needle or probe 110 above a second vial, where a subsequent sample will be aspirated. This process can continue for all vials in a carrier.

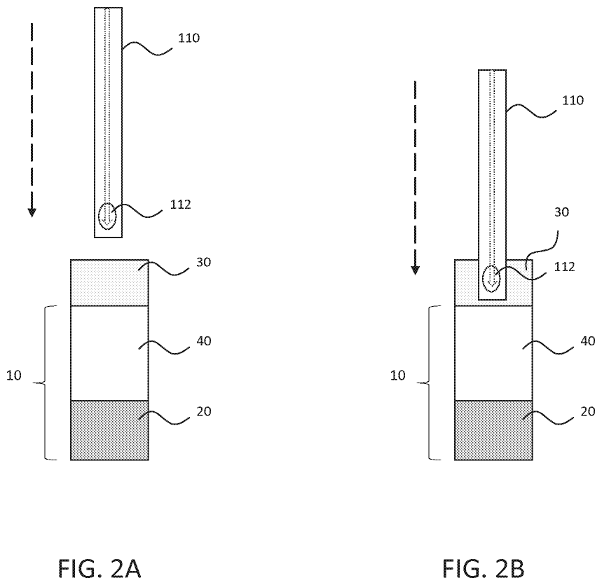

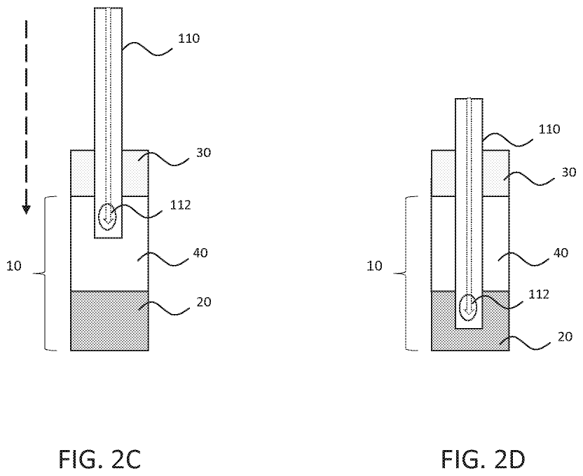

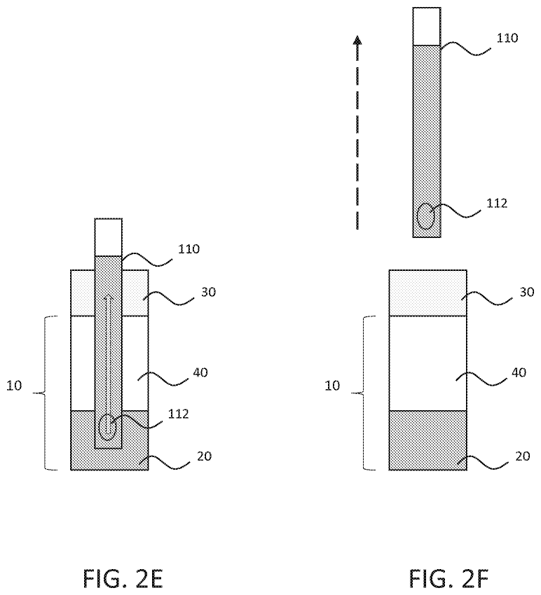

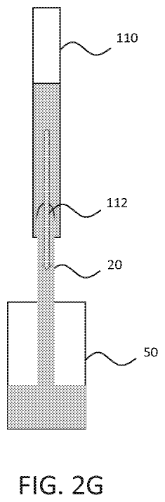

FIGS. 2A-2G show an exemplary sequence of aspiration, dispensing, and cleansing by the aspiration system 100, according to embodiments. As shown in FIG. 2A, the tip of the probe is above the stopper 30 before it is further lowered. The lowering of the probe 110 (or raising) described below is caused by the motor 190 under control of the controller 180. During descent, the aspiration system 100 may cause gas (e.g., air) to be emitted through the probe 110 and outwardly through the distal aperture 112. The emissions are illustrated with dotted lines. A single emission may have a relatively small volume of air and may last for between, for example, about 20 to 25 ms. The emissions are also referred to herein as “puffs”. As shown in FIG. 2A, the controller 180 controls the pump 160 to emit air into the chamber 130. The pressure in the chamber 130 corresponds to the pressure at the distal aperture 112. The increase in pressure in the chamber 130 causes an increase in pressure in the probe 110, such that puffs of air are emitted through the distal aperture 112. As puffs are emitted through the distal aperture 112, the controller 180 assesses the level of the signal provided by the transducer 150, which generates a variable output signal that corresponds to the sensed pressure in the chamber 130. The pressure sensed by the transducer 150 may be assessed by the controller 180 during and/or after the duration of the puff. Thus, the controller 180 detects pressure associated with the distal aperture 112. In the case of FIG. 2A, the transducer 150 senses a relatively low pressure (e.g., atmospheric pressure), and the controller 180 causes the probe 110 to descend to lower elevations towards the stopper.

The controller 180 may further control the pump 160 to suck the volume of puffed air out of the aspiration system 100 after the puff and after the pressure measured by the transducer 150 has been assessed by the controller 180.

Throughout the descent or ascent of the probe 110, the controller 180 tracks how much the motor 190 has moved—e.g., the controller 180 keeps track of the number of steps that the motor 190 has turned. As the original elevation of the tip of the probe 110 (and the elevation of the distal aperture 112) can be known, the controller 180 can infer the elevation of the tip of the probe 110 or distal aperture 112 by counting the positive and/or negative steps.

As shown in FIG. 2B, as the controller 180 controls the motor 190 to lower the probe 110, the tip of the probe 110 penetrates the stopper 30. Puffs of air continue to be emitted during the descent (again, under control of the controller 180 via the pump 160). Eventually, the distal aperture 112 becomes partially, and then fully blocked by the stopper 30 as the probe 110 is lowered under control of the controller 180. During the descent, the controller 180 continues to assess the sensed pressure provided by the transducer 150. As the distal aperture 112 becomes blocked by the stopper 30 (partially and/or fully blocked), the pressure in the chamber 130 increases as puffs are emitted (i.e., the pump 160 creates pressure in the chamber 130, but the air is not emitted through the distal aperture 112, thereby increasing the pressure in the chamber 130). When the controller 180 determines that the sensed pressure has increased (e.g., increased above a threshold differential from the pre-penetration sensed pressure), the controller 180 infers that the distal aperture 112 has an elevation such that it is in the stopper 30. The controller 180 may sense a gradual increase in pressure as the distal aperture 112 progressively passes partially into the stopper 30, and when the pressure has stabilized, the controller 180 can infer that the distal aperture 112 is fully in the stopper. The controller 180 may ascertain the height of the stopper 30 by tracking the operations of the motor 190. The controller 180 may compare the ascertained height of the stopper 30 with a predetermined height to determine whether the stopper 30 is at the expected height.

The controller 180 may control operations of the probe 110 once the distal aperture 112 enters or is embedded within the stopper 30. For example, the controller 180 may adjust the rate of descent by controlling the motor 190 appropriately. For example, before the distal aperture 112 enters the stopper 30, the descent may be controlled to be relatively rapid. Once the distal aperture 112 enters the stopper 30, the descent may be controlled to be slower. This may be advantageous because proceeding more slowly may improve the accuracy of the measurement of the sample level, as discussed below. More air puffs and pressure readings may be performed when the motor 190 causes descent at a slower speed. In such a case, the rate and duration of puffs may be constant at the different speeds of the motor 190. Alternatively, the controller 180 may further cause the duration and/or rate of puff emissions to change by controlling the pump 160.

As shown in FIG. 2C, as the probe 110 continues to lower, the distal aperture 112 extends through the stopper 30 and into the gas 40 in the vial 10 (either fully or partially). Using the puff/measuring techniques described above, the controller 180 detects a decrease in pressure (e.g., by measuring a change in pressure, a decreasing pressure, a stabilized pressure, an absolute pressure value, and/or the like). At this time, the controller 180 infers that the distal aperture 112 has extended past the stopper 30 into the gas 40 by determining an absolute pressure or a decrease in pressure over a threshold. In the upper region of the vial 10, immediately below the stopper 30, there is no sample and only gas (e.g., air). At this time, the controller 180 can determine whether the pressure in the gas 40 in the vial 10 is at a desired pressure (e.g., atmospheric pressure). If it is not (too high or too low), the controller 180 can cause the vent 162 or pump 160 to allow for fluid communication between the chamber 130 and the external environment, such that the pressure in the chamber 130 (and therefore the pressure in the probe 110 and the pressure in the gas 40 inside the vial 10) equilibrates with the pressure in the external environment. Alternatively, the controller 180 may cause equilibration via the vent 162 irrespective of the detected pressure in the gas 40. This may be beneficial, as the pressure of the gas 40 in the vial 10 may be above or below a desired pressure, and as such, the volume of the sample 20 that is aspirated may be imprecise. By equilibrating the pressure of the gas 40 in the vial 10, the accuracy of the aspirations may be improved.

As the aspiration system 100 may be able to equilibrate pressure in the gas 40 via the vent 162, there may be no need to create apertures between the probe 110 and the stopper 30 via grooves or another technique. This technique may enable the exterior surface of the probe 110 to be substantially smooth, such that there is an insubstantial or no gap between the stopper 30 and the exterior surface of the probe 110. This may reduce or eliminate coring, such that no pieces of the stopper 30 (or an insubstantial amount of pieces) fall into the vial 10.

As discussed above, once the distal aperture 112 has entered the gas 40 in the vial 10, (fully or partially) the rate of descent of the probe 110 may be changed under control of the controller 180. For example, the rate of descent may increase to the rate before the distal aperture 112 entered the stopper 30.

As shown in FIG. 2D, as the descent of the probe 110 continues, the distal aperture 112 enters the sample 20, either partially or fully. At that time, the controller 180 may sense an increase in pressure using similar techniques as those discussed above with respect to FIGS. 2B and 2C. Once the distal aperture 112 is inferred by the controller 180 to be in the sample 20 (e.g., completely submerged in the sample 20), then the descent of the probe 110 can be halted.

For a given volume of the sample 20 and the known dimensions of the vial 10, the height of the upper surface of the sample 20 is known. Having kept track of the elevation of the distal aperture 112 during the descent of the probe (e.g., by counting steps performed by the motor 190) controller 180 can infer the actual height of the sample 20 and compare it to the expected height of the sample 20. If the actual height of the sample 20 is lower than expected, then the controller 180 can infer a “short-sample” condition. If a short-sample condition is determined, then the controller 180 can take further action, such as alert the user, abort the analysis, or the like. Similarly, the controller 180 can infer if there is more of a sample 20 than is expected. Thus, by monitoring or assessing the operation of the motor 190 over time (e.g., counting steps when the motor 190 is a stepper motor) and by detecting the pressure associated with the distal aperture 112, an elevation of the distal aperture 112 can be associated relative to the sample 20. For example, the elevations of the distal aperture 112 and the upper surface of the sample 20 can be absolutely determined, and thus the relative elevations would be known as well.

In some examples, the controller inferring or determining that the distal aperture is in the sample can include scenarios such as the controller using a predetermined pressure threshold or pressure change threshold where exceeding this threshold causes subsequent steps to be taken (e.g., counting a particular number of motor steps to place the aspiration needle in the sample and aspirating once a certain number of steps are met). In other words, such inference or determination is not limited to a particular state of the sample (e.g., an actual determination that the aspiration needle is in the sample), but that particular conditions are met (e.g., pressure thresholds or pressure change thresholds) which would cause subsequent controlled or programmed steps to take place (e.g., once a pressure or pressure change threshold is met or exceeded, an aspiration needle is subsequently lowered in to the sample by a certain number of motor steps, and then aspiration occurs).

As shown in FIG. 2E, once the distal aperture 112 has entered the sample 20, then a desired volume of the sample 20 can be aspirated. To perform aspiration, the controller 180 causes the pump 160 to create a partial vacuum in the chamber 130. The controller 180 can assess the actual pressure in the chamber 130 via the transducer 150. A given pressure in the chamber 130 may cause a known amount of the sample 20 to be aspirated through the probe 110 and optionally into the reservoir 120 if the volume of the aspirated portion of the sample 20 is large enough. The rate of pumping determines the rate of aspiration. The controller 180 can calculate and control the amount of volume of gas pumped into the chamber 130 and/or the rate of pumping according to the desired volume of aspirated sample and/or rate of aspiration. A volume of sample that is aspirated may be, for example, between 100 to 120 μL. This volume can be sufficient for analysis of the sample by multiple analysis processes.

According to an embodiment, the probe 110 can be lowered to a predetermined elevation (e.g., a predetermined number of steps) according to the amount of the sample 20 to be aspirated. In an illustrative example, every millimeter of height in the sample 20 can correspond to 100 μL of volume. If 200 μL of the sample 20 is to be aspirated, then the distal aperture 112 can be lowered to an elevation of about 2 mm (or greater) below the upper surface of the sample 20 (e.g., the detected upper surface of the sample, as described with respect to FIG. 2D) before aspiration begins. Again, the elevation of the distal aperture 112 can be inferred or determined by keeping track of the operations of the motor 190 (e.g., counting steps).

It may be desirable to position the distal aperture 112 relatively close to the upper surface of the sample 20, such as between about 2 to 4 mm (e.g., 3 mm) below the upper surface of the sample 20. This may be advantageous to minimize carryover or avoid aspiration of bubbles above surface level.

According to an embodiment, only one aspiration is performed in a given aspiration cycle. According to another embodiment, the aspiration system 100 progressively aspirates the sample 20. A portion of the sample 20 can be aspirated when the distal aperture 112 is at a first elevation. The controller 180 can detect when the portion of the sample 20 at the first elevation has been aspirated. For example, the transducer 150 can continue to obtain measurements during aspiration. This information can be used by the controller 180 to maintain the pressure in the probe 110. When a spike or rapid change of pressure is detected, this can be indicative that aspiration is complete (e.g., at least an intended amount of the sample 20 has been aspirated). The probe 110 can then be lowered, such that the distal aperture 112 moves to a second elevation below the first elevation. Another portion of the sample 20 can then be aspirated through the distal aperture 112 through a similar process. This process can be repeated any suitable number of times while lowering the probe 110 until the desired volume of the sample 20 has been aspirated.

As shown in FIG. 2F, the probe 110 is moved upwardly until the distal tip of the probe 110 is above the stopper 30. The controller 180 can infer the location of the distal tip of the probe 110, again, by tracking operations of the motor 190 (e.g., counting steps, and in this case, reverse or negative steps) and accounting for the length of the probe 110. As discussed above, the controller 180 can cause the probe 110 to move at different speeds, such as the speeds discussed above, but in an upward direction. Instead of using puffs to determine the location of the distal tip of the probe 110, the controller 180 can track the operations of the motor 190 to determine the elevation of the distal tip of the probe 110.

As shown in FIG. 2G, the aspirated portion of the sample 20 is dispensed. Once the probe 110 has been pulled out, the aspiration system 100 or the vial 10 may be moved to a new location. The probe 110 may then be aligned with a container 50, in which the aspirated portion of the sample 20 is dispensed through the distal aperture 112 and into the container 50. The sample 20 that is dispensed is used in a subsequent process, such as an analysis process (e.g., impedance measuring, hematology flow imaging, or the like). The controller 180 can control the dispensation process by causing the pump 160 to pump air into the chamber 130. The amount of air pumped into the chamber 130 affects the volume of the sample 20 that is dispensed through the distal aperture 112. The controller 180 can calculate and control an amount of aspirated sample for dispensation. The controller 180 may further control the rate of dispensation by controlling the rate of air pumped by the pump 160 into the chamber 130.

All or a portion of the aspirated sample may be dispensed into the container 50, or different portions of the aspirated sample may be dispensed into different containers. In the latter case, again, the controller 180 can calculate and control the amount of aspirated sample to be dispensed by causing the pump 160 to pump a specific volume of air into the chamber 130. After dispensing the aspirated portion of the sample 20 into the container 50 (or multiple containers), the probe 110 may become aligned with a different vial to perform the aspiration and dispensation process again.

The interior of the lumen in the probe 110 as well as the reservoir 120 is cleaned using a cleaning fluid. The valve to the port 170 is opened (e.g., caused to be opened by the controller 180) such that cleaning fluid can flow through the port 170, into the reservoir 120, and down through the probe 110 exiting the distal aperture 112. Once the cleaning fluid (or a sufficient amount thereof) has flowed into the reservoir 120 and probe 110, the valve for the port 170 can be closed (for example, under control of the controller 180). The controller 180 may cause the vent 162 to be opened to allow the cleaning fluid to flow, and/or the controller 180 may cause the pump 160 to increase the pressure in the chamber 130 to cause the cleaning fluid to flow through the probe 110 and out of the distal aperture 112. A wash collar may surround the exterior of the probe 110 to perform external cleaning as high vacuum applied by the wash collar removes the waste as the probe 110 is moved vertically through the wash collar. Further, the cleaning fluid form within the probe 110 may exit through the distal aperture 112 and be removed into the wash collar.

The cleaning process can be performed periodically. For example, the cleaning process can be performed before the aspiration system 100 transitions to aspirating a sample from a subsequent vial. This may prevent contamination of the sample in the new vial from the sample in the previous vial.

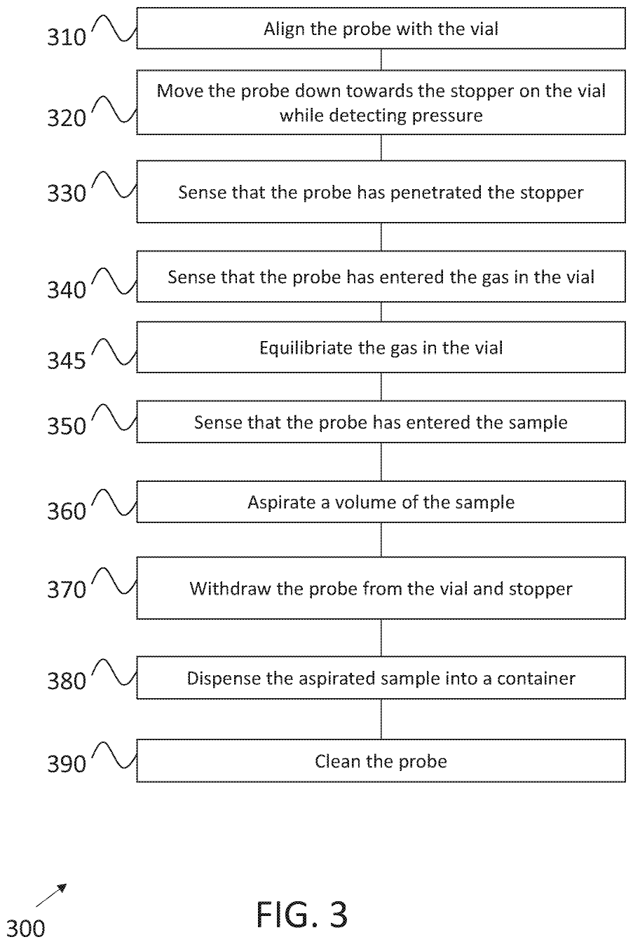

FIG. 3 is a flowchart 300 for a method of aspirating a sample 20 from a vial 10. The following description of flowchart 300 is with respect to aspiration system 100, but is not so limited. Portions or the entirety of the steps in flowchart 300 may be executed or caused by the controller 180. The controller 180 can execute a set of instructions stored on a non-transitory computer readable medium to execute or cause the steps of flowchart 300. Steps may be performed overlappingly, simultaneously, or in a different order, as will be understood.

At step 310, the probe 110 is aligned with the vial 10 above the stopper 30. The controller 180 can align the probe 110 with the vial 10, for example, by controlling an x-y gantry to move the probe 110 into the proper location. The valve at the port 170 is set to the closed state to isolate pressure within the aspiration system 100. The aspiration system 100 generates puffs of gas through the distal aperture 112, such as the puffs described above. The puffs can be caused by the controller 180 controlling the pump 160 as discussed above. The region above the stopper 30 may be gas (e.g., air) at atmospheric pressure or another suitable pressure. The transducer 150 senses the pressure in the chamber 130, and the controller 180 receives a signal from the transducer 150 and assesses the pressure in the chamber 130. The sensed pressure is that of the external environment at step 310. The controller 180 tracks the operation of the motor 190, for example, by tracking the number of steps up and/or down executed by the motor 190. The controller 180 may include a counter that counts the steps as the flowchart 300 continues. The controller 180 can determine the maximum elevation of the probe 110 based on feedback from the motor 190, as described above. When the probe 110 is at its maximum elevation, the controller 180 can reset the counter so it is at zero or some predetermined value. The aspiration system 100 may sense the elevation(s) of the probe 110 through other means, such as via positional sensor(s) (not shown).

At step 320, the probe 110 begins its descent towards the stopper 30. Step 320 corresponds to FIG. 2A. The descent is caused by the controller 180 controlling the motor 190. During descent, puffs through the distal aperture 112 continue as discussed above. The controller 180 continues to recognize that the pressure at the distal aperture 112 is that of the external environment. The controller 180 continues to assess the motor 190 (e.g., by increasing the step counter according to each step the controller 180 causes the motor 190 to execute). Thus, the controller 180 can infer the height of the distal aperture 112.

At step 330, the probe 110 is lowered and penetrates the stopper 30, and distal aperture 112 enters the stopper 30 (either partially or entirely). Step 330 corresponds to FIG. 2B. When the distal aperture 112 is at the elevation of the stopper 30, the controller 180 determines that there has been an increase in pressure in the chamber 130 due to the puffs through the distal aperture 112 being blocked, either partially or entirely.

At step 340, the probe 110 continues to be lowered until the distal aperture 112 is at an elevation of the gas 40 within the vial 10. Step 340 corresponds to FIG. 2C. The controller 180 continues to evaluate the elevation of the distal aperture 112. The controller 180 senses that the pressure in the aspiration system 110 has dropped, and infers that the distal aperture 112 is in the gas 40.

At step 345, the controller 180 controls the vent 162 to equilibrate the pressure in the gas 40 with that of the external environment. The pressure in the gas 40 may be higher or lower than that of the external environment. For example, in the case of a blood sample, when blood is drawn from a patient, the gas 40 may have a pressure lower than that of the external environment. As another example, if the stopper 30 is placed on the vial 10 after the sample 20 is in the vial 10, then that may cause the gas 40 to have a pressure that is higher than that of the external environment. After equilibration, the controller 180 can cause the vent 162 to close, such that the pressure in the aspiration system 110 is isolated from the pressure in the external environment.

At step 350, the distal aperture 112 is continued to be lowered until it enters the sample 20 (either partially or completely). Step 350 corresponds to FIG. 2D. The controller 180 continues to track the elevation of the distal aperture 112. Once the distal aperture 112 has entered the sample 20, the controller 180 detects an increase in pressure. The controller 180 can then infer that the distal aperture 112 has entered the sample 20, and further infer the height of the upper surface of the sample 20. The distal aperture 112 can then be lowered to a predetermined elevation below the upper surface of the sample 20.

At step 360, the controller 180 causes the pump 160 to create a vacuum in the chamber 130 to aspirate a predetermined volume of the sample 20 into the probe 110 and/or reservoir 120. Step 360 corresponds to FIG. 2E.

At step 370, after aspiration in step 360 is complete, the controller 180 causes the motor 190 to withdrawn the probe 110 from the vial 10 and the stopper 30. Step 370 corresponds to FIG. 2F.

At step 380, the controller 180 causes the pump 160 to add pressure into the chamber 130 to cause the aspirated sample (e.g., aliquot) to be dispensed into the container 50. Step 380 corresponds to FIG. 2G.

At step 390, the controller 180 causes cleaning fluid to be dispensed (and/or aspirated through a portion of the aspiration needle (e.g., probe 110 and reservoir 120) and out of the distal aperture 112. The cleaning fluid may then be sucked up by a wash collar around the exterior of the probe 110. In this manner, the residual aspirated sample can be washed out of the aspiration needle.

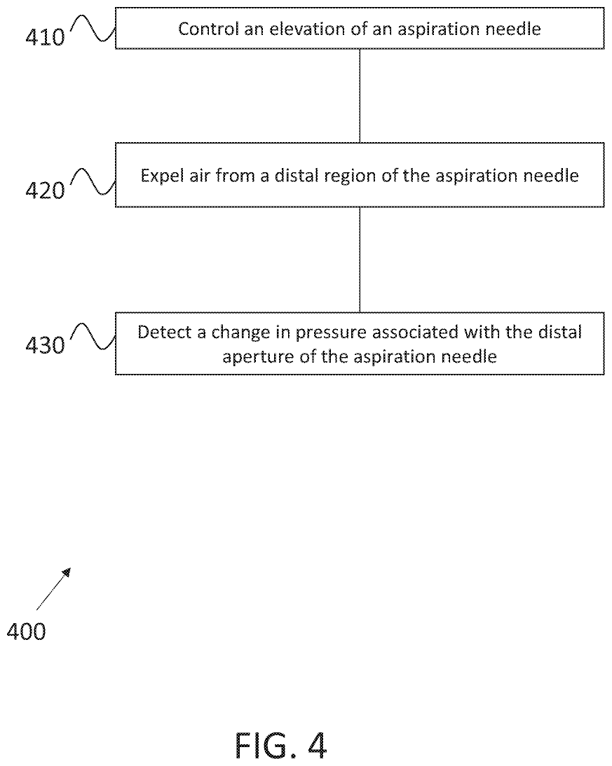

FIG. 4 is a flowchart 400 for a method of aspirating a sample 20 from a vial 10. The following description of flowchart 400 is with respect to aspiration system 100, but is not so limited. Portions or the entirety of the steps in flowchart 400 may be executed or caused by the controller 180. The controller 180 can execute a set of instructions stored on a non-transitory computer readable medium to execute or cause the steps of flowchart 400. Steps may be performed overlappingly, simultaneously, or in a different order, as will be understood.

At step 410, an elevation of an aspiration needle (inclusive of the probe 110) is controlled. The elevation of the aspiration needle is controlled by the controller 180 and the motor 190, as described above. The aspiration needle can aspirate a blood sample 20 from a specimen vial 10. The vial 10 can further contain a gas, which may be at a different pressure than the environmental pressure (e.g., either higher or lower). The aspiration needle includes a distal aperture 112. The aspiration needle may equilibrate the gas 40 to the environmental pressure during aspiration.

At step 420, air is expelled from a distal region of the aspiration needle, which may be the distal aperture 112 of the probe 110. Air may be expelled via the air pump 160 as controlled by the controller 180, as described above.

At step 430, a change in pressure associated with the distal aperture 112 of the aspiration needle is detected. The pressure may be detected by the transducer 150 in combination with the controller 180, as described above.

The operations described herein may be performed or effected using a controller (e.g., controller 180) having hardware, software, and/or firmware. The various method steps may be performed in conjunction with modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single controller board or separated into different controller boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like.

All patents, patent publications, patent applications, journal articles, books, technical references, and the like discussed in the instant disclosure are incorporated herein by reference in their entirety for all purposes.

Different arrangements of the components depicted in the drawings or described above, as well as components and steps not shown or described are possible. Similarly, some features and sub-combinations are useful and may be employed without reference to other features and sub-combinations. Embodiments of the present disclosure have been described for illustrative and not restrictive purposes, and alternative embodiments will become apparent to readers of this patent. In certain cases, method steps or operations may be performed or executed in differing order, or operations may be added, deleted or modified. It can be appreciated that, in certain aspects of the present disclosure, a single component may be replaced by multiple components, and multiple components may be replaced by a single component, to provide an element or structure or to perform a given function or functions.

It will be understood by those skilled in the art that various changes may be made and equivalents may be substituted without departing from the scope of the novel techniques disclosed in this application. In addition, many modifications may be made to adapt a particular situation or material to the teachings of the novel techniques without departing from its scope. Therefore, it is intended that the novel techniques not be limited to the particular techniques disclosed, but that they will include all techniques falling within the scope of the appended claims.

1. An aspiration system for a biological analyzer, comprising:
an aspiration needle configured to aspirate a blood sample from a specimen vial, wherein the aspiration needle includes a distal aperture;
a motor operatively connected to the aspiration needle, the motor configured to control an elevation of the aspiration needle;
an air pump configured to expel air from a distal region of the aspiration needle; and
a controller configured to detect a change in pressure associated with the distal aperture of the aspiration needle.

2. The aspiration system of claim 1, wherein the specimen vial further contains a gas having a pressure different from an environmental pressure, and wherein the aspiration system is configured to equilibrate the pressure of the gas to the environmental pressure.

3. The aspiration system of claim 1, wherein the controller is configured to:
determine that the distal aperture of the aspiration needle has reached the blood sample by detecting an increase in pressure; and
allow aspiration to occur when the distal aperture of the aspiration needle is below a surface of the blood sample.

4. The aspiration system of claim 1, wherein the aspiration needle includes a chamber in fluid communication with the air pump.

5. The aspiration system claim 4, further comprising a membrane between the chamber and the distal aperture of the aspiration needle, wherein the membrane is gas-permeable, and wherein the membrane is not substantially permeable by the blood sample or a cleaning fluid.

6. The aspiration system of claim 4, wherein the controller includes a pressure transducer configured to measure a pressure within the chamber.

7. The aspiration system of claim 6, wherein the aspiration needle includes a probe, and wherein the controller is configured to calculate a height of the blood sample based on a number of motor steps used to lower the probe to the blood sample, and is further configured to detect a short-sample condition when an elevation of the probe exceeds a predetermined elevation based on a predetermined volume of the blood sample.

8. The aspiration system of claim 7, wherein the probe has an exterior surface without grooves.

9. The aspiration system of claim 1, further comprising a vent configured to allow pressure equilibration between a pressure within the aspiration needle and atmospheric pressure, and wherein the controller is configured to calculate a required vacuum in the aspiration needle to aspirate or dispense a specific volume of the blood sample when the pressure is equilibrated.

10. The aspiration system of claim 1, wherein the controller is configured to detect when the distal aperture of the aspiration needle is in a stopper on the specimen vial according to a detected increase in pressure, or has passed through the stopper and into a region of air within the specimen vial above the blood sample.

11. An aspiration system for a biological analyzer, comprising:
an aspiration needle configured to aspirate a blood sample from a specimen vial, wherein the aspiration needle includes a distal aperture;
a motor operatively connected to the aspiration needle, the motor configured to control an elevation of the aspiration needle;
a controller configured to:
detect a pressure associated with the distal aperture of the aspiration needle to associate an elevation of the distal aperture relative to the blood sample;
cause the motor to lower the elevation of the aspiration needle ; and
after the aspiration needle is lowered, cause aspiration of a predetermined volume of the blood sample.

12. The aspiration system of claim 11, wherein the controller is further configured to detect a change in pressure associated with the distal aperture to determine that the distal aperture is in the blood sample before aspirating the blood sample.

13. The aspiration system of claim 12, wherein the controller is further configured to determine a short-sample condition based on an elevation of the blood sample.

14. The aspiration system of claim 11, wherein the motor is a stepper motor, and wherein the controller is further configured to count a number of steps that the motor moves as the elevation of the aspiration needle changes to associate the elevation of the distal aperture relative to the blood sample.

15. The aspiration system of claim 14, wherein the controller is further configured to, after the elevation of the distal aperture is associated with the blood sample, lower the aspiration needle by a predetermined number of steps according to a predetermined volume of the blood sample to be aspirated.

16. The aspiration system of claim 11, wherein the aspiration system is configured to equilibrate a pressure of a gas in the specimen vial to an environmental pressure before aspirating the blood sample.

17. The aspiration system of claim 11, further comprising:
an air pump configured to cause air to be expelled from the distal aperture and to create a vacuum to aspirate the blood sample through the distal aperture; and
a pressure transducer configured to measure the pressure associated with the distal aperture when air is being expelled from the distal aperture.

18. The aspiration system of claim 17, further comprising a vent configured to allow pressure equilibration between a pressure within the aspiration needle and atmospheric pressure, and wherein the controller is further configured to calculate a required vacuum in the aspiration needle to aspirate or dispense a specific volume of the blood sample when the pressure is equilibrated.

19. The aspiration system of claim 17, wherein the aspiration needle includes a chamber in fluid communication with the air pump, and wherein the pressure transducer is configured to measure a pressure within the chamber.

20. The aspiration system claim 19, further comprising a membrane between the chamber and the distal aperture of the aspiration needle, wherein the membrane is gas-permeable, and wherein the membrane is not substantially permeable by the blood sample or a cleaning fluid.
